(19) United States
(12) Patent Application Publication
(10) Pub. No.: US 2011/0059113 A1
(43) Pub. Date: Mar. 10, 2011

(54) GAB2 AMPLIFICATION IN MELANOMA

(75) Inventor: Julide Tok CELEBI, New York, NY (US)

(73) Assignee: The Trustees Of Columbia University In The City Of New York, New York, NY (US)

(21) Appl. No.: 12/909,093

(22) Filed: Oct. 21, 2010

Related U.S. Application Data
(63) Continuation-in-part of application No. PCT/US2009/033511, filed on Feb. 9, 2009.
(60) Provisional application No. 61/046,589, filed on Apr. 21, 2008.

(51) Int. Cl. A61K 39/395; A61K 31/7105; A61K 31/713; A61P 35/00; C12Q 1/02; C12Q 1/68; C40B 30/04

(52) U.S. Cl. 424/172.1; 514/44 A; 435/29; 435/6; 506/9

(57) ABSTRACT

Provided herein are methods of detecting or inhibiting melanoma growth based on Gab2 protein expression. Gab2 protein was found to be either amplified and/or overexpressed in melanoma. Gab2 protein expression correlated with clinical melanoma progression and higher levels of expression were seen in metastatic melanomas compared to primary melanoma and melanocytic nevi. Over-expression of Gab2 potentiates, whereas silencing of Gab2 reduces, migration and invasion of melanoma cells. Gab2 mediated hyperactivation of Akt signaling in the absence of growth factors and inhibition of the PI3K-Akt pathway decreased Gab2-mediated tumor cell migration and invasive potential. Gab2 over-expression resulted in enhanced tumor growth and metastatic potential in vivo. These results demonstrate a previously undefined role for Gab2 in melanoma tumor progression and metastasis.

This application is a Continuation-in-part of Int'l App'l No. PCT/US2009/033511, filed Feb. 9, 2009, which claims benefit of U.S. Ser. No. 61/046,589, filed Apr. 21, 2008. The contents of the preceding application are hereby incorporated in its entirety by reference into this application.

### BACKGROUND OF THE INVENTION

The GAB genes, encoding mammalian GAB1, GAB2, and GAB3, represent a family of scaffolding or docking proteins (1). They contain several functional motifs that mediate interactions with other signaling molecules. GAB2 (Grb2-associated binding protein 2) interacts with the adapter protein GRB2 and this interaction serves as a bridge between GAB2 and receptor tyrosine kinases such as FGFR, c-Met, EGFR, IGF-1R, and NGFR. Upon stimulation, GAB2 undergoes tyrosine phosphorylation, creating a number of docking sites to mediate interactions with SH2 domain-containing proteins such as the tyrosine phosphatase SHP2, p85 subunit of PI3K, PLCγ, CRK, and SHC. Association with these molecules is critical for the function of GAB proteins in mediating intracellular signaling pathways from the receptors. The interaction of GAB2 with SHP2 activates RAS-ERK signaling, whereas its association with the p85 subunit of PI3K is crucial in mediating the PI3K-AKT signaling (2).

### GAB2 in Cancer

In addition to its important roles in several signaling pathways, there is evidence that GAB2 might a play role in oncogenic transformation, such as Bcr-Abl transformation GAB2−/− cells expressing Bcr-Abl exhibit defective PI3K-AKT and ERK activation, which likely reflects the inability of Bcr-Abl to signal to p85 and SHP2, respectively, via GAB2 (9).

There is a growing body of evidence on the role of GAB2 in breast carcinogenesis. GAB2 expression is very low in normal and immortalized breast epithelial cells. It is overexpressed in a subset of breast cancer cell lines (10). GAB2 is tyrosine phosphorylated following stimulation with heregulin or EGF in MCF-7 human breast cancer cells, and overexpression of GAB2 in MCF-10 cells increases cell proliferation and alters growth factor dependency (11). GAB2 overexpression alone can increase the proliferative capacity of mammary epithelial cells. In transgenic mice, overexpression of GAB2 accelerates Her2/Neu induced mammary tumorigenesis by activating SHP2-ERK pathway (12). In mice, ablation of GAB2 severely suppresses lung metastasis suggesting a prominent role for GAB2 in promoting mammary tumor metastasis (13). In p27kip1 deficient breast cancer cells, GAB2 and AKT are increased, leading to significant enhancement of cell migration and invasion, and to lung metastasis in nude mice (8).

GAB2 is located on 11g13.5-14.1, a region amplified in 10-15% of human breast cancers (14). In a cohort consisting of 142 breast carcinomas, GAB2 gene upregulation, evaluated by microarrays, was seen in 8% of the cases (12). GAB2 amplification correlated with overexpression in these samples. Taken together, these findings suggest that amplification of GAB2, in combination with other genetic abnormalities, may contribute to the genesis of breast tumors with 11q13.5-14.1 amplification.

### The RAS-ERK Pathway in Melanoma

The RAS-ERK pathway is critical in melanoma and regulates cell fate decisions such as proliferation, survival, migration and differentiation, with ERK being hyperactivated in up to 90% of melanomas. In melanoma, ERK can be activated by mutational activation of growth-factor receptors such as KIT (˜2-3% of melanomas) (3, 4), or more commonly through gain-of-function mutations in NRAS (˜10% of melanomas) or BRAF (˜50% of melanomas) (5). There are, however, melanomas wild-type for NRAS, BRAF, and KIT suggesting other genes or mechanisms leading to constitutive ERK activation. In preliminary studies, amplifications of GAB2 were identified in ˜10% of melanomas, some of which are wild-type for NRAS, BRAF, and KIT. It is of interest to test whether GAB2 amplification is an additional event in melanoma leading to constitutive ERK activation or whether GAB2 cooperates with mutant NRAS or BRAF. As a novel genetic event in melanoma, it is of interest to understand the relationship of GAB2 amplification with the currently known oncogenes in the RAS-ERK pathway. These studies will help dissect the mechanisms for ERK activation in melanoma that will provide basis for novel targeted therapies.

### The PI3K-AKT Pathway in Melanoma

The phosphoinositide-3-OH kinase (PI3K) signaling regulates cell survival, proliferation, cell motility, invasion and metastasis and it is hyperactivated in a high proportion of melanomas (6). This is explained in part by the findings that PIK3CA mutations occur in 3% of metastatic melanomas, PTEN function is lost in between 5% to 20% of late stage melanomas, and AKT is overexpressed in up to 60% of melanomas. In three-dimensional melanoma cultures, ERK and PI3K signaling must both be inhibited to suppress cell growth, demonstrating that both pathways are important (7). In breast cancer cells, AKT activation through GAB2 results in migration, invasion and metastasis (8). Data provided herein provide evidence that GAB2 activation promotes migration, invasion, increased tumor growth and metastasis in mice. Since GAB2 activation is critical for PI3K-AKT signaling, it is of interest to test whether GAB2 amplification or overexpression activates this pathway to synergize with constitutively activated RAS-ERK signaling, and contribute to invasiveness of melanoma cells.

### Significance

Melanoma is currently the sixth most common cancer in the U.S. with incidence rates faster than for any other cancer. The lifetime risk of developing invasive melanoma in the U.S. is currently 1 in 71 compared with an estimate of 1 in 600 in 1960 (15). Melanoma is the second leading cause of lost productive years and it is the most common cancer among women 20 to 29 years of age (16). If diagnosed early it can be cured by surgery. However metastatic disease has a poor prognosis with a median survival of 6-9 months (16). There are no therapies available for metastatic melanomas that have a significant impact on survival.

The studies presented herein are the first steps towards a better understanding of the contribution of GAB2 to melanoma pathogenesis. It is expected to extend the current knowledge on molecules that contribute to invasive and metastatic melanoma phenotype and lead to understanding of intricate pathways that control these processes. These studies are essential for development of effective treatments against metastatic melanoma. Since NRAS and BRAF are validated therapeutic targets in melanoma, drugs that target this pathway are of considerable interest. Although some of the clinical trials are ongoing, to date, none have showed significant effectiveness for the treatment of metastatic melanoma. Therefore, identifying novel therapeutic targets and developing new treatment strategies are needed.

### SUMMARY OF THE INVENTION

In an attempt to identify genes that contribute to melanoma pathogenesis, a genome-wide search using BAC array CGH and SNP arrays identified increased copy numbers of Gab2 located on 11g14.1. Gab2 is an adaptor molecule that potentiates activation of the Ras-Erk and PI3K-Akt pathways and has recently been implicated in human cancer. As shown below, it was found that Gab2 was either amplified (˜10%) and/or overexpressed (˜50%) in melanoma. Gab2 protein expression correlated with clinical melanoma progression and higher levels of expression were seen in metastatic melanomas compared to primary melanoma (p=0.0137) and melanocytic nevi (p=0.004). Overexpression of Gab2 potentiates, whereas silencing of Gab2 reduces, migration and invasion of melanoma cells. Gab2 mediated hyperactivation of Akt signaling in the absence of growth factors and inhibition of the PI3K-Akt pathway decreased Gab2-mediated tumor cell migration, invasive and metastatic potential. Gab2 overexpression resulted in enhanced tumor growth and metastatic potential in vivo. These studies demonstrate a previously undefined role for Gab2 in melanoma tumor progression and metastasis.

### BRIEF DESCRIPTION OF THE DRAWINGS

FIG. 1 shows GAB proteins are implicated in regulation of the RAS-ERK pathway through their ability to bind to and activate the SH2 domain containing protein-tyrosine phosphatase SHP2, as well as in activation of the PI3K-AKT pathway, by virtue of their ability to bind to the p85 regulatory subunit of PI3K, thereby activating the associated p110 catalytic subunit.

FIG. 2 shows identification of increased copy numbers of 11q13.5-14.1 in melanoma. A: Hierarchial clustering of DNA copy number data from melanoma tumor samples and cell lines shows subclusters delineating two independent amplicons on 11q13.2 and 11q13.5 14.1, harboring cyclin D1 and Gab2 respectively. All of the 64 tumor samples consisted of metastatic melanomas. Of the 20 melanoma cell lines, 5 were derived from primary (WM39, WM1552, WM793, WM278, and WM35) and 15 from metastatic melanomas. B: 11q14.1 DNA copy number data showing focal amplifications with Gab2 at the center of the amplicon. C: A representative case showing Gab2 locus amplification by FISH. D: SNP signal intensity colorgram verifying the 11q13.5-14.1 amplicon in melanoma samples. E: Gene expression analysis of Gab2 by qRT-PCR in tumors with increased copy numbers of 11q14.1 shows consistent upregulation of Gab2 (means±SD, N=3, p<0.05).

FIG. 3 shows Gab2 is overexpressed in metastatic melanoma. Total cell lysates were isolated and Gab2 levels were measured by Western blot analysis. β-actin was used as a loading control. Gab2 expression was determined by densitometry and was normalized with β-actin. The BRAF and NRAS mutation status of the cell lines is indicated. All melanoma cell lines with BRAF mutation harbor the BRAF^{V600E} mutation. WM1361 cell line has the NRAS^{Q61R} mutation. Gab2 is expressed at very low levels, if any, in a normal human melanocyte cell line and primary melanoma cells, whereas it is expressed at high levels in metastatic cell lines.

FIG. 4 shows Gab2 expression correlates with clinical tumor progression. A: Quantitative analysis of Gab2 protein expression (AQUA-based analysis) in a series of nevi, primary melanomas, and metastatic melanomas is shown. Although the variance is large, there are significantly higher expression levels of non-nuclear Gab2 in metastatic lesions compared to both primary melanomas and nevi. The score for each case is the average of two observations from two unique tissue spots of a single block tissue microarray. Data shown in this figure are from 18 nevi and 15 primary tumors and 15 metastatic tumors. B: Representative examples of primary and metastatic melanoma immunostaining of Gab2 are shown.

FIG. 5 shows Gab2 promotes tumor cell migration and invasion in vitro. A, B: Western blot analysis, Migration and Matrigel Invasion assays of control (scrambled) or Gab2 knockdown with siRNA of metastatic melanoma cell lines (Mewo, A2058 and Ht144 cells) are shown. Gab2 knockdown resulted in decreased migration (means±SD, N=3). Gab2 silencing resulted in a significant reduction of the invasive ability of the cells (means±SD, N=3, *p<0.05). C, D: Western blot analysis of forced Gab2 expression in primary melanoma cell lines (WM793, WM278, WM1552, and WM3862) as well as Migration and Matrigel Invasion Assay results are shown. Forced expression of Gab2 in primary melanoma cells resulted in a significant increase of melanoma cell migration and invasion (means±SD, N=3, *p<0.05). E: Representative examples of vector and Gab2expressing primary melanoma cells (WM793) invading through the Matrigel are shown.

FIG. 6 shows Gab2 enhances tumor growth and metastatic capability of melanoma cells in vivo. A: In vivo tumor growth of WM3862 primary melanoma cell line expressing either vector or Gab2 was determined. Mice were injected with 1×10⁶ cells expressing either Gab2 or vector alone subcutaneously. Tumors were measured with a caliper in two dimensions and mice were sacrificed when tumor surface area reached 1.5 cm². Kaplan Meier curves show that mice injected with Gab2-overexpressing cells reached tumor burden threshold faster than the vector group (*p<0.05). All mice injected with Gab2 overexpressing cells reached the tumor burden threshold between days 16 to 21, whereas mice injected with vector alone reached this threshold between days 26 to 46. B: Representative tumor sections stained with H&E and Gab2 (by immunohistochemistry) are shown. Note that Gab2 overexpression alters melanoma cell morphology from epitheloid to spindle in subcutaneous tumors. C: In vivo metastasis of WM3862 cell line expressing either vector or Gab2 was evaluated. Mice were injected with vector or Gab2 overexpressing WM3862 cells via the tail veins. Seven out of 11 (63%) mice in the Gab2-overexpressing group developed lung metastasis whereas none in the vector group had lung tumors (*p<0.05). D: Representative examples of lung sections stained with H&E and Gab2 are shown. Note that Gab2-expressing tumor cells form nodules within the lung and invade into the lung parenchyma.

FIG. 7 shows Gab2 promotes tumor cell migration and invasion via Akt activation. A: Gab2 silencing in Mewo metastatic melanoma cell line using siRNA technology shows decreased Akt phosphorylation. B: Western blot analysis showing vector or Gab2 forced expression into primary melanoma cell lines WM793, WM278, WM1552, and WM3862 after serum starvation for 24 hours. Gab2 overexpression results in Akt and PDK1 phosphorylation in melanoma cells. C: Treatment of Gab2-overexpressing primary melanoma cells with the PI3K inhibitor, LY294002 (25 μM), for 24 hours results in a significant decrease in tumor cell migration and invasion potential (means±SD, N=3, *p<0.05).

FIG. 8 shows Gab2 enhances in vivo tumor growth. Mice were injected with 1×10⁶ WM3862 cells expressing either Gab2 or vector alone subcutaneously. A: Tumor volume measurement of each mouse is shown on day 16 after injection. M1 (mouse 1) injected with Gab2 overexpressing cells was sacrificed on day 16 due to increased tumor burden. All mice injected with cells overexpressing Gab2 had significantly increased tumor volume as compared to those injected with vector alone on day 16, the last day in which all mice were alive in both groups (*p<0.05). B: Representative examples of the xenografts are shown (arrows point to tumors). Note that WM3862 cells expressing vector alone give rise to pigmented tumors in SCID mice (top panel), whereas WM3862 cells overexpressing Gab2 are amelanotic (lower panel).

FIG. 9 shows GAB2 amplifications in melanoma by array CGH. Supervised hierarchical clustering of copy number data shows clustering among melanomas arising from sun-protected sites (acral and mucosal melanoma subtypes).

FIG. 10 shows representative examples of cases with GAB2 amplifications that are wild type for BRAF, NRAS, and KIT. These cases show high levels of GAB2 protein expression by immunohistochemistry. Note the absence of staining of the overlying epidermis as a negative control (top panel).

FIG. 11 shows frequency distribution of genetic alterations in GAB2, KIT, BRAF and NRAS among two groups of melanoma. Sun-protected sites include cases occurring on acral sites (palms and soles) and mucous membranes. Sun-exposed sites include melanomas arising from head, neck, trunk and extremities. BRAF and NRAS mutations were mutually exclusive. Two cases, one occurring on sun-protected and the other on sun-exposed sites, showed an increased copy number in both GAB2 and KIT. ** indicates statistically significant association of GAB2 amplification with melanomas arising from sun-protected sites (acral and mucosal melanomas) (P=0.005).

FIG. 12 shows the study results on the potential impact of GAB2 as a molecular prognostic marker for melanoma.

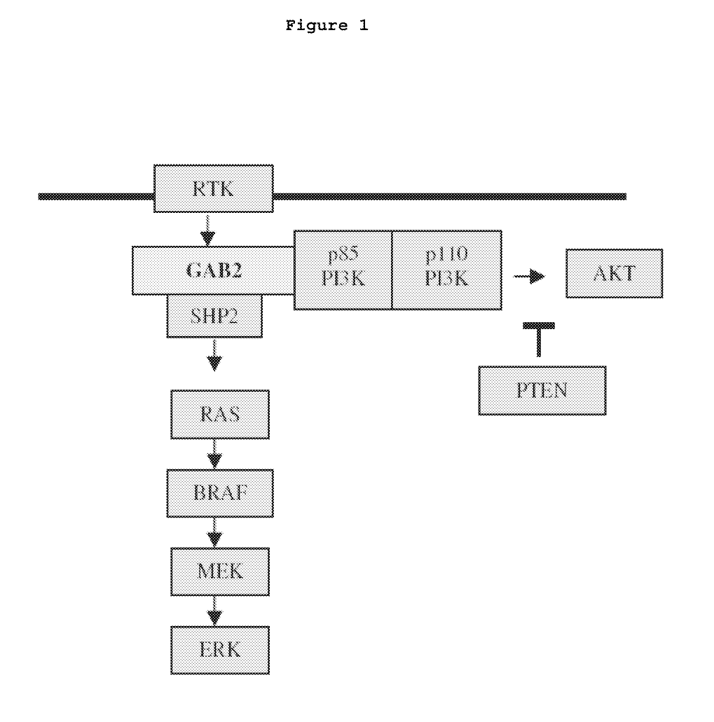

### DETAILED DESCRIPTION OF THE INVENTION

The present invention provides a method of detecting melanoma cells in a subject, comprising the steps of: determining Gab2 (Grb2-associated binding protein 2) expression level in a tissue sample from the subject; and comparing the Gab2 expression level to that in a tissue sample from a normal subject, wherein increased Gab2 expression would indicate presence of melanoma cells in the subject. The Gab2 expression level would further help determine the stage of melanoma and predict the subject's prognosis and outcome. For example, increased Gab2 expression would indicate poor prognosis or poor chance of survival for the subject. In general, a tissue sample includes, but is not limited to, a skin biopsy sample. The above method can be used to detect invasive melanoma cells, metastatic melanoma cells, acral melanoma cells, or mucosal melanoma cells. One of ordinary skill in the art would readily determine Gab2 expression at the protein, mRNA or DNA level. For example, increased Gab2 expression may be demonstrated by increased Gab2 protein expression, increased DNA copy number, or GAB2 gene amplification according to standard procedures in the art.

The present invention also provides a method of inhibiting the development, progression, or metastasis of melanoma cells in a subject, comprising the step of administering to the subject an agent that decreases, modifies or inhibits Gab2 expression, wherein decreased, modified or inhibited Gab2 expression would inhibit the development, progression, or metastasis of melanoma cells in the subject. In general, Gab2 expression is inhibited or decreased at the protein, mRNA or DNA level. Examples of anti-Gab2 agents include, but are not limited to, anti-Gab2 antibody, anti-sense Gab2, small interfering RNA, small hairpin RNA, or microRNA against Gab2.

As used herein, anti-Gab2 antibody refers to polyclonal, monoclonal, and other forms of recombinant polypeptide (e.g. single chain variable fragment, humanized antibody etc.) that binds to Gab2 protein. Such polyclonal/monoclonal anti-Gab2 and other forms of recombinant anti-Gab2 polypeptides can be readily generated.

As used herein, anti-sense Gab2 refers to anti-sense RNA or DNA sequences that inhibit Gab2 expression.

As used herein, small interfering RNA refers to RNA oligo sequences that inhibit Gab2 expression.

As used herein, small hairpin RNA refers to RNA hairpin sequences that inhibit Gab2 expression.

As used herein, microRNA against Gab2 refers to microRNA sequences that inhibit Gab2 expression.

The present invention also provides a method of inhibiting the development, progression, or metastasis of melanoma cells in a subject, comprising the step of administering to the subject an agent that inhibits cellular signaling mediated by Gab2, wherein inhibited cellular signaling mediated by Gab2 would inhibit the development, progression, or metastasis of melanoma cells in the subject. Examples of signaling mediated by Gab2 include Ras-Erk signaling or PI3K-Akt signaling.

The present invention also provides a method for identifying candidate anti-Gab2 agents which inhibit or modify the function of Gab2 protein, wherein said method comprises (a) obtaining a melanoma sample; (b) contacting candidate agents with the melanoma sample; and (c) assaying one or more activities mediated by Gab2 protein, wherein decreased Gab2-mediated activities in the presence of the candidate agents as compared to control indicates that the candidate agents are anti-Gab2 agents. Examples of Gab2-mediated activity include, but are not limited to, Gab2-mediated tumor cell migration or invasive potential, Ras-Erk signaling, or PI3K-Akt signaling. In one embodiment, the method is a high throughput screening method. High throughput screening is a widely practiced method in the art, and in view of the disclosure provided herein, one of ordinary skill in the art would readily apply high throughput screening to screen for anti-Gab2 agents.

The invention will be better understood by reference to the Experimental Details which follow, but those skilled in the art will readily appreciate that the specific experiments detailed are only illustrative, and are not meant to limit the invention as described herein, which is defined by the claims which follow thereafter.

Throughout this application, various references or publications are cited. Disclosures of these references or publications in their entireties are hereby incorporated by reference into this application in order to more fully describe the state of the art to which this invention pertains. It is to be noted that the transitional term “comprising”, which is synonymous with “including”, “containing” or “characterized by”, is inclusive or open-ended and does not exclude additional, un-recited elements or method steps.

### EXAMPLE 1

### GAB2-Mediated Signaling Promotes Melanoma Metastasis

Metastatic melanoma is a disease with a poor prognosis currently lacking effective treatment. Critical biologic features of metastasis include acquisition of migratory competence, growth factor independence, and invasive potential. In an attempt to identify genes that contribute to melanoma pathogenesis, a genome-wide search using BAC array CGH and SNP arrays in a series of 64 metastatic melanoma samples and 20 melanoma cell lines identified increased copy numbers of Gab2 located on 11q14.1. In this study, it is found Gab2 was either amplified (˜11%) and/or overexpressed (˜50%) in melanoma. Gab2 protein expression correlated with clinical melanoma progression and higher levels of expression were seen in metastatic melanomas compared to primary melanoma (p=0.0137) and melanocytic nevi (p=0.004). It is found that overexpression of Gab2 potentiates, whereas silencing of Gab2 reduces, migration and invasion of melanoma cells. Gab2 mediated hyperactivation of Akt signaling in the absence of growth factors and inhibition of the PI3K-Akt pathway decreased Gab2-mediated tumor cell migration and invasive potential. Gab2 overexpression resulted in enhanced tumor growth and metastatic potential in vivo. These studies demonstrate a previously undefined role for Gab2 in melanoma tumor progression and metastasis.

### Materials and Methods

Tumor Specimens and Cell Lines. Sixty-four metastatic melanoma frozen tissue samples were obtained from the tissue banks of the Department of Pathology at Columbia University and the Department of Cancer Genetics at University of Münster, Fachklinik Hornheide, Germany. Tumors were examined histopathologically and those with at least 90% neoplastic tissue were selected. In some samples where tumor cells were admixed with a prominent lymphocytic infiltrate, cells were microdissected using the PixCell II Laser Capture Microdissection System™ (Arcturus, Mountain View, Calif.). Twenty-three cell lines derived from primary and metastatic melanomas were obtained from the ATCC and the Wistar Institute and cultured according to their instructions. A normal human melanocyte cell line was generated from neonatal foreskin as described (10) and maintained in melanocyte growth medium-4 with supplements and growth factors (Lonza Inc, Allendale, N.J.). The protocol was approved by Columbia University's Institutional Review Board.

BAC Array CGH. Arrays with ˜19,000 RPCI-11 BAC clones providing an average resolution of 150 kb, were used. A complete list of the RPCI-11 BAC clones on the 19K array can be found at: http://microarrays.roswellpark.org. BAC array CGH was performed as described previously. (11) Briefly, 2 μg of reference and test sample genomic DNA were fluorescently labeled using the BioArray CGH Labeling System™ (Enzo Life Sciences, Inc., New York, N.Y.). The DNA was denatured in the presence of random primer at 99° C. for 10 minutes, cooled to 4° C., labeled by adding dNTP-cyanine 3 mix (or dNTP-cyanine 5) and Klenow, and incubated overnight at 37° C. Hybridization to the 19K arrays was performed at 55° C. for 16 hours using a GeneTAC™ hybridization station (Genomic Solutions, Inc., Ann Arbor, Mich.). The hybridized slides were scanned with a Genepix™ 4200A scanner (Molecular Devices, Inc., Sunnyvale, Calif.) and image analysis was performed with ImaGene™ software (Biodiscovery, Inc., Elsegundo, Calif.). Log₂ ratios were computed and normalized using sub-grid loess. Clones were ordered by chromosomal position according to the University of California Santa Cruz human genome sequence build 36.1 (http://genome.ucsc.edu).

SNP Array. SNPs were genotyped and the copy numbers were estimated using 250K arrays (Affymetrix, Santa Clara, Calif.). Genomic DNA was cleaved with the restriction enzyme, Sty I, ligated with linkers, followed by PCR amplification. The PCR products were purified and digested with DnaseI to a size ranging from 250 to 2,000 bp. Fragmented PCR products were then labeled with biotin and hybridized to the array. Arrays were washed on the Affymetrix™ fluidics stations. The bound DNA was then fluorescently labeled using strepavidin-phcoerythrin conjugates and scanned using the Gene Chip Scanner 3000™ (Affymetrix, Santa Clara, Calif.). DChip™ software was used for SNP array data analysis as described previously. (12) Data was normalized to baseline array with median signal intensity at the probe intensity level using the invariant set normalization method. A model-based (PM-MM) method was used to obtain the signal values for each SNP in each array. To infer the DNA copy number from raw signal data, the Hidden-Markov model was used based on the assumption of diploidy for normal samples. Mapping information of SNP locations and cytogenetic band were based on curation of Affymetrix™ and University of California Santa Cruz. A cutoff of >2.8 copies in more than three consecutive SNPs were defined as amplification.

Quantitative Real-time PCR. cDNA was synthesized using Superscript First Strand Synthesis™ kit as per instructions of the manufacturer (Invitrogen, Carlsbad, Calif.). One μg total RNA, 10 mM dNTP mix and 50 μM oligo(dT)₂₀ were incubated at 65° C. for 5 min, placed on ice for 1 min and cDNA Synthesis mix was added. The reaction was incubated at 50° C. for 50 min and terminated at 85° C. for 5 min. Real-time PCR was performed with 100 ng input RNA per reaction containing 1×TaqMan™ universal PCR Master mix and the appropriate TaqMan™ probe on a 7300 Real-Time PCR™ system (Applied Biosystems, Foster City, Calif.). Gab2 (Hs00373045) and beta-Actin (4326315E) TaqMan™ probes were purchased from Applied Biosystems™. All samples were prepared in triplicates on each plate and at least three plates were analyzed. Relative mRNA levels were determined using the Comparative CT Method.

Fluorescent in situ Hybridization (FISH). RP11-653J20 and RP11-444N24 BAC clones were obtained from Invitrogen™. DNA was prepared from BAC clones using standard methods and labeled by nick-translation using spectrum red or spectrum green dUTP fluorochromes. Spectrum red or spectrum greenlabeled centromeric probes were used to enumerate chromosome numbers (Vysis, Downers Grove, Ill.. Hybridization signals were scored on at least 20 metaphase spreads of cell lines or 200 interphase nuclei from tissue sections on DAPI counterstained slides.

Immunofluorescence and Automated Quantitative Analysis (AQUA) of Tissue Microarrays. For protein expression, tissue microarrays containing specimens obtained from melanocytic nevi (18), primary melanomas (15) and metastases (15) were used. Immunofluorescence staining of tissue microarrays was performed as described previously (13) using an anti-Gab2 antibody (Upstate, Charlottesville, Va.). The AQUA™ software linked to the fluorescence microscopy system was used as described previously (13) for quantification of the Gab2 protein within the tumor region of each tissue microarray core.

Immunoblotting and Antibodies. Cells were lysed with the PhosphoSafe™ extraction reagent (Novagen, San Diego, Calif.) containing protease and phosphatase inhibitors. Protein quantity was determined by Bradford assay (Bio-Rad Laboratories, Inc., Hercules, Calif.). Proteins were separated by SDS gel electrophoresis under reducing conditions and then transferred to 0.45 mm nitrocellulose membranes by electroblotting. The membranes were blocked with 5% nonfat powdered milk in PBS and then probed with primary antibodies at a 1:1000 dilution in TBST with 5% milk. The membranes were then washed with TBST and probed with horseradish peroxidase-conjugated secondary antibodies at 1:2000 dilutions in TBST with 5% milk. After washing in TBST, the membranes were developed with the ECL™ Western blotting detection system (Pierce Biotech, Rockford, Ill.). The films were scanned using the Molecular Dynamics Personal Densitometer SI™ and the protein band density was quantified using the ImageQuant™ software (Molecular Dynamics, Sunnyvale, Calif.). The following commercial antibodies were used in this study: Gab2 (Upstate, Charlottesville, Va.); Gab1, phospho-Akt (Ser-473), total Akt, phospho-PDK1, total PDK1 (Cell Signaling, Danvers, Mass.); and Actin (Santa Cruz, Santa Cruz, Calif.).

Gene Knockdown using siRNA. Small interfering RNA against Gab2 and control (scrambled) RNAs were purchased from Dharmacon (Dharmacon, Lafayette, Colo.). 1.5×10⁵ cells were transfected with 300 pmol/L of siRNA using Oligofectamine reagent in Opti-MEM I reduced serum medium (Invitrogen, Carlsbad, Calif.). Four hours later, the medium was replaced with culture medium supplemented with 10% fetal bovine serum. Forty-eight hours later the cells were harvested for immunoblotting.

Generation of Gab2-expressing Stable Cell Lines. Primary melanoma cell lines (WM793, WM278, WM1552, and WM3862) expressing either Gab2 or vector were generated using retroviral transduction as described previously. (14,15) Gab2 full-length cDNA, obtained from Origene™, was subcloned into pBABE-puro retroviral vector (Addgene, Cambridge, Mass.). Phoenix-Eco (ecotropic) packaging cells were transiently transfected with 20 ug of pBABE-puro retroviral vector by CaPO4 co-precipitation method. The packaged viruses released by the Phoenix-Eco cells were used for infecting AM12 (amphotropic) packaging cells in order to generate stable clones of virus producing cells and to achieve high viral titers. (14) Forty-eight hours following infection, the cells were split into puromycin selection medium (2 μg/ml) and surviving colonies were selected. Expression levels of Gab2 were evaluated by Western blot analysis of AM12 clones. Viral supernatant was then collected from these producer cells over a 24-48 hour period, filtered through a 0.45 mm filter to remove cell debris and used for infecting the melanoma cells. Lysates from infected cells were subjected to Western blot analysis using an antibody against Gab2.

Migration and Invasion Assays. Cell migration was assessed by adding 1×10⁵ cells into the upper chamber of a motility chamber system containing 8-μm pores (modified Boyden chamber assay, BD Biosciences, San Jose, Calif.). Invasion assays were performed by seeding 1×10⁵ cells into Biocoat Matrigel invasion chambers (BD Biosciences, San Jose, Calif.) in serum-free medium, and by adding 10% FBS to the lower wells as chemoattractant. For both assays, the cells were incubated for 24 hours, the filters were stained with crystal violet, and the number of cells that penetrated through the filter was counted under a light microscope at 20× magnification. For each membrane, the mean number of cells in three randomly selected fields was determined. For each assay, three independent experiments were performed in which the bars represent the mean±SD.

In Vivo Tumorigenicity and Metastasis Assays. Tumor growth was evaluated by subcutaneous injection of 1×10⁶ vector or Gab2 expressing stable cell lines into the flanks of 7 week-old female severe combined immunodeficient (SCID) mice (N=4 in each group). Tumor length and width were measured using calipers. Mice were sacrificed when tumor surface area reached 1.5 cm² and tumors were harvested and examined histologically. For experimental metastasis assays, vector or Gab2-expressing stable cell lines were injected into SCID mice via the tail veins (1×106 cells per mouse, N=12 in each group). Mice were sacrificed 3 weeks after inoculation, the lungs were harvested and examined histologically. Animal experiments were performed under a protocol approved by the Columbia University's Institutional Committee for Care and Use of Animals.

Statistical analysis. Statistical analyses were performed with a t-test in which p<0.05 was considered as statistically significant. Differences between the groups in the experimental metastasis assays were analyzed using Fisher's exact test.

### Results

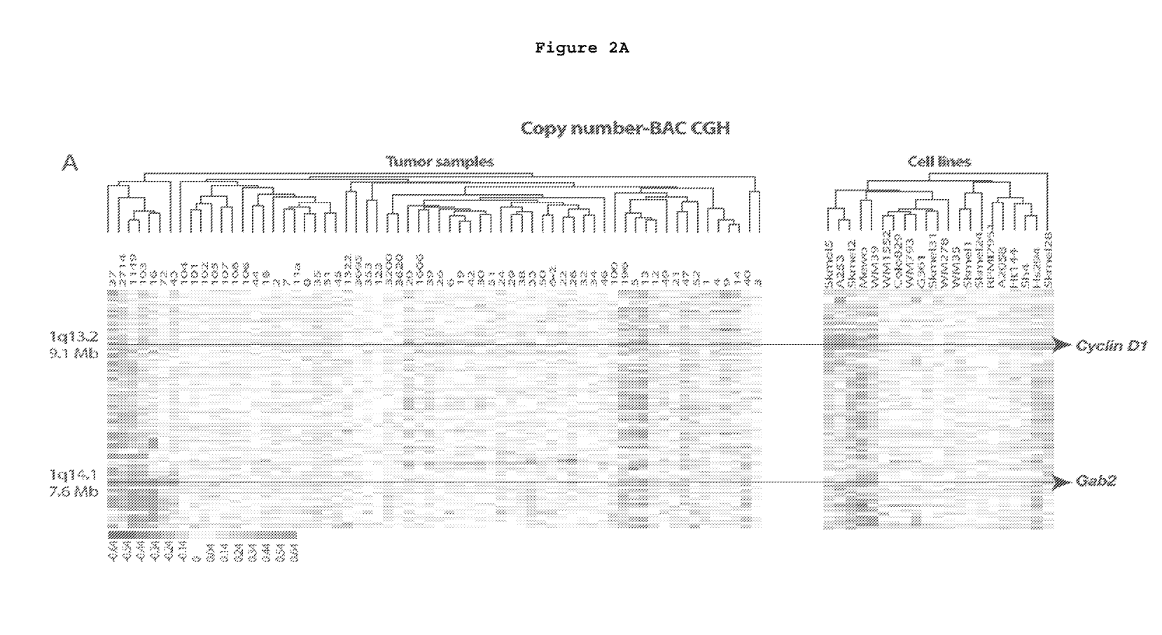

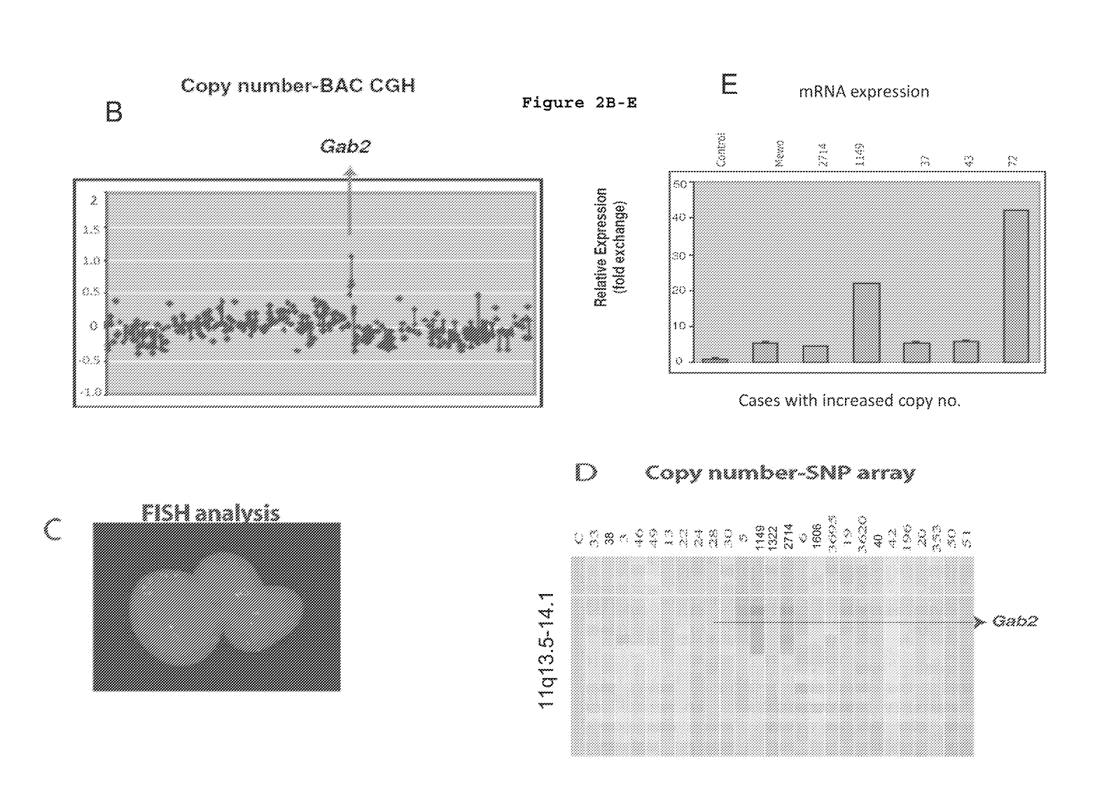

Increased DNA Copy Numbers of Gab2 in Metastatic Melanoma. In order to identify genes critical for melanoma pathogenesis, BAC array CGH and SNP arrays were used to search for genome-wide copy number alterations in 64 metastatic melanoma samples and 20 melanoma cell lines. Several distinct amplicons were found on chromosome 11q. Among them, increased DNA copy numbers of the cyclin D1 locus on 11q13.2, previously described for melanoma, was present in 5 of the 84 (6%) samples (1/64 tumors, 5/20 cell lines). More importantly, there is a distinct amplicon, independent of cyclin D1, in 9 of the 84 (11%) samples (7/64 tumors, 2/20 cell lines), in which consistent focal amplifications with Gab2 gene at the center of the amplicon were observed (FIG. 2A, B, D). These data were further confirmed by FISH analysis (FIG. 2C). Six of the nine cases were available for evaluation of Gab2 mRNA expression by qRT-PCR, which showed consistent upregulation in all six cases compared to a human melanocyte cell line and non-amplified tumor samples (FIG. 2E). Identification of increased DNA copy numbers of Gab2 among metastatic melanomas along with gene amplification in a subset of tumors resulted in further analysis of Gab2 in melanoma.

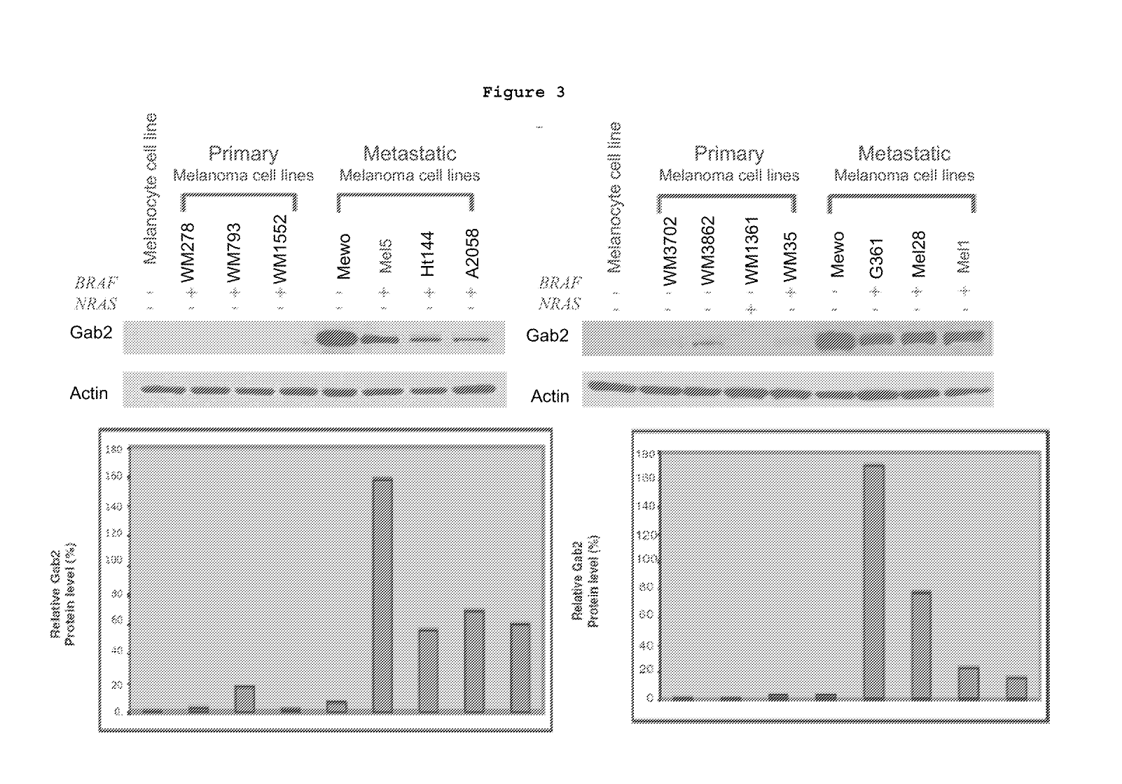

Gab2 is Overexpressed in Metastatic Melanoma. The expression profile of Gab2 in a normal human melanocyte cell line and a panel of melanoma cell lines were examined by Western blot analysis. Gab2 protein was expressed at low levels in the melanocyte cell line and in primary melanoma cells whereas high level Gab2 expression was observed in metastatic cell lines (FIG. 3).

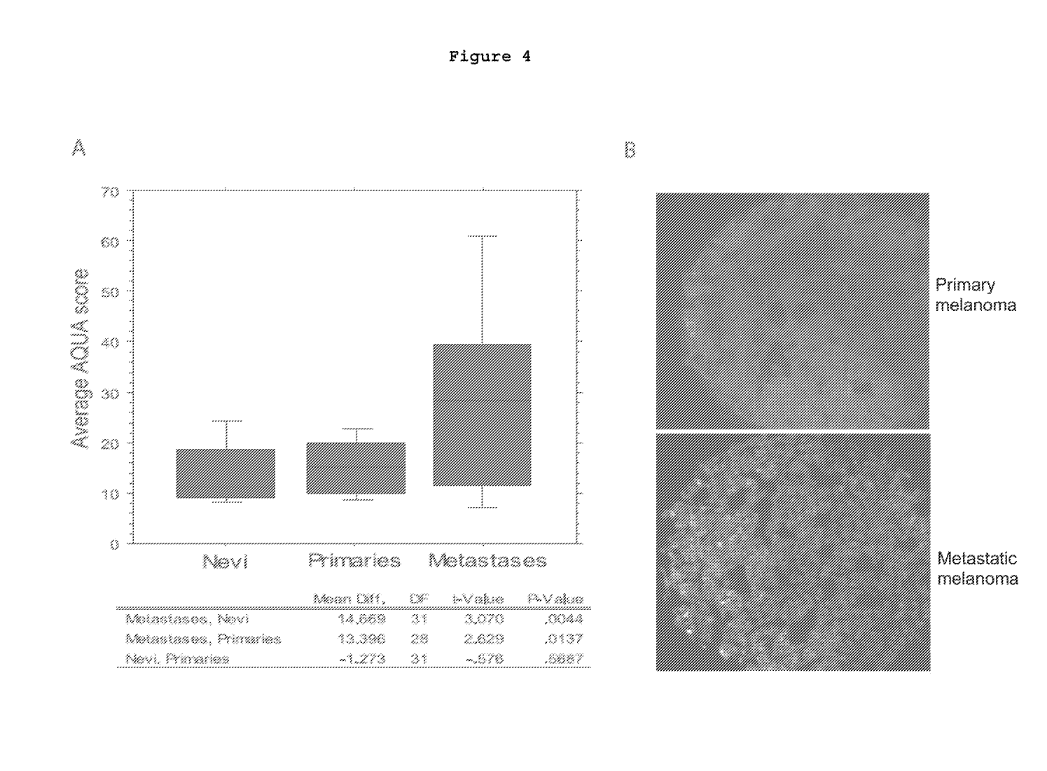

To confirm the above results and to investigate the expression profile of Gab2 during clinical melanoma progression, the analysis was extended by studying an independent series of melanocytic tumor samples, including melanocytic nevi, primary and metastatic melanomas. For these studies, automated quantitative analysis (AQUA) system was used, which allows for rapid analysis of immunofluorescence on tissue microarrays, reduces the human variability of scoring by eye and results in a continuous range of protein expression rather than ordinal (0, +1, +2, and +3) scores. It also allows for quantification of the protein of interest within user-defined subcellular compartments within the tumor region of each tissue microarray spot. Specifically, AQUA analysis of Gab2 protein expression was performed on 18 melanocytic nevi, 15 primary and 15 metastatic melanoma samples each represented by two tissue microarray spots. Results showed that Gab2 protein is expressed at significantly higher levels in metastatic melanomas compared to melanocytic nevi (p=0.0044) and primary melanomas (p=0.0137), identifying Gab2 as a molecular marker of neoplastic progression (FIG. 4).

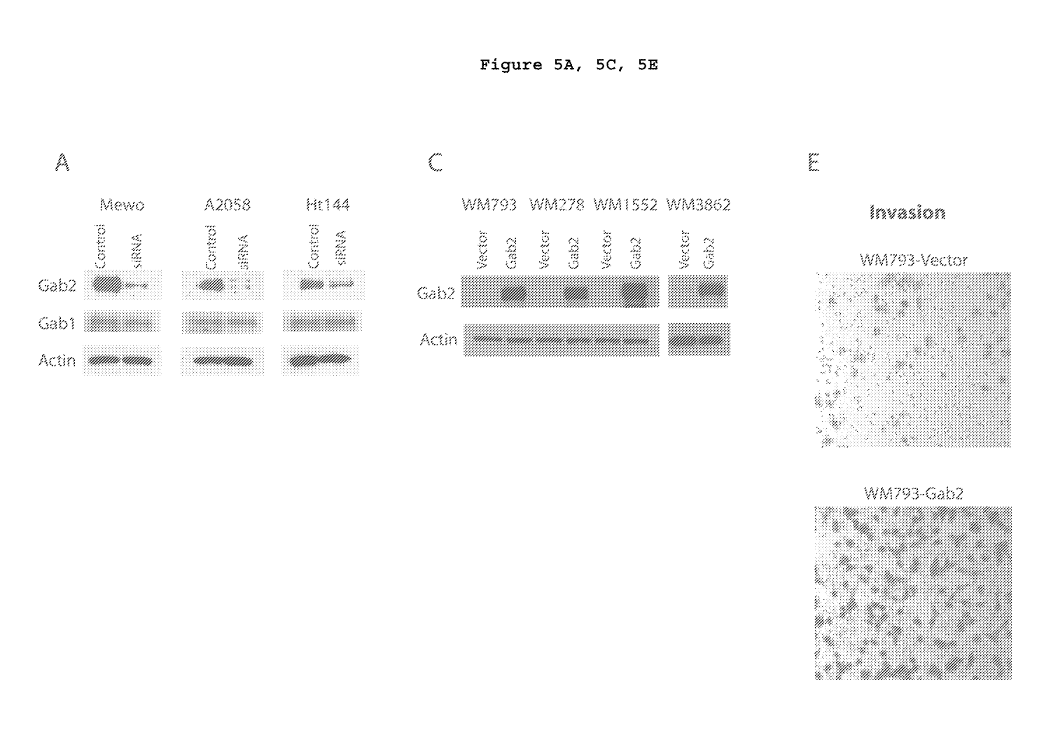

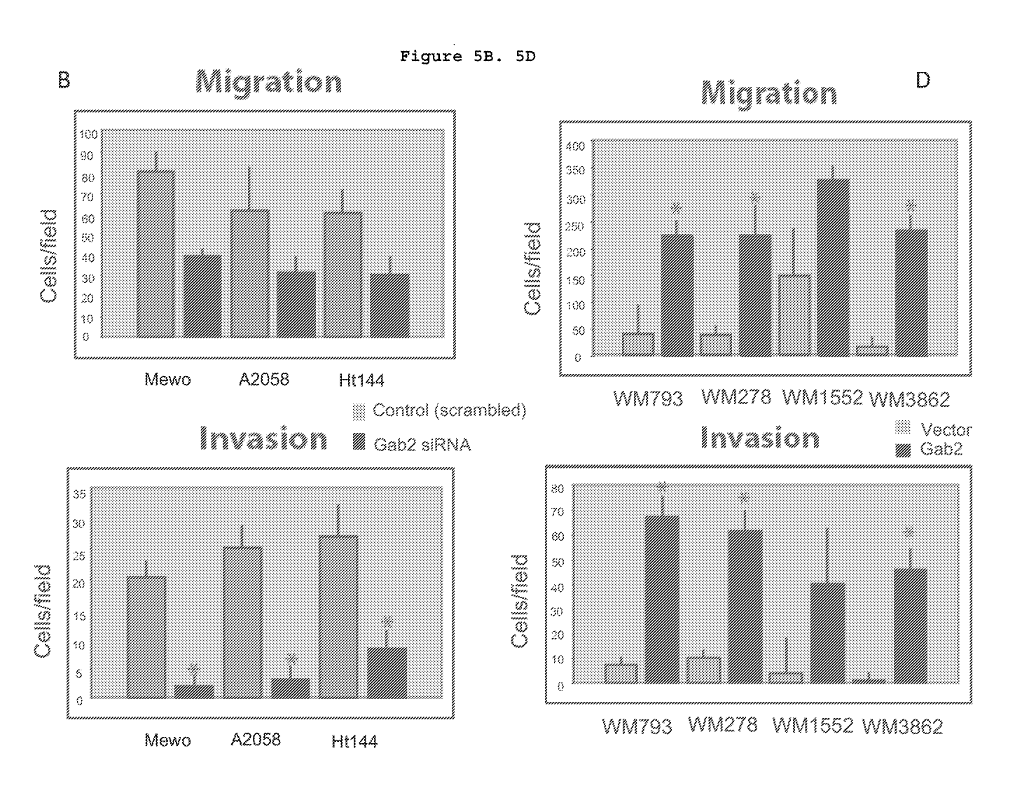

Gab2 Promotes Migration and Invasion of Melanoma Cells. To investigate the role of Gab2 in tumor progression, siRNA technology was used to downregulate Gab2 in three metastatic melanoma cell lines that overexpress Gab2; Mewo, Ht-144 and A2058. Gab2 silencing did not affect Gab1 expression (FIG. 5A). As shown in FIG. 5B, Gab2 knockdown decreased the migratory potential of tumor cells and led to a significant decrease in their invasive capacity (p<0.05), suggesting that continuous Gab2 expression is required for in vitro invasion of metastatic melanoma cells. It is next determined whether Gab2 overexpression could enhance migration and invasiveness of primary melanoma cells. For these experiments, WM793, WM278, WM1552, and WM3862 cells that were derived from primary melanomas and have low or undetectable levels of Gab2 were used. All of these cell lines except WM3862 cells harbor the BRAFV600E mutation. Forced expression of Gab2 in these cells led to a significant enhancement of migration and invasion (p<0.05) (FIG. 5 C, D and E). Taken together, these studies show that Gab2 is capable of enhancing motility and invasive capabilities of primary melanoma cells.

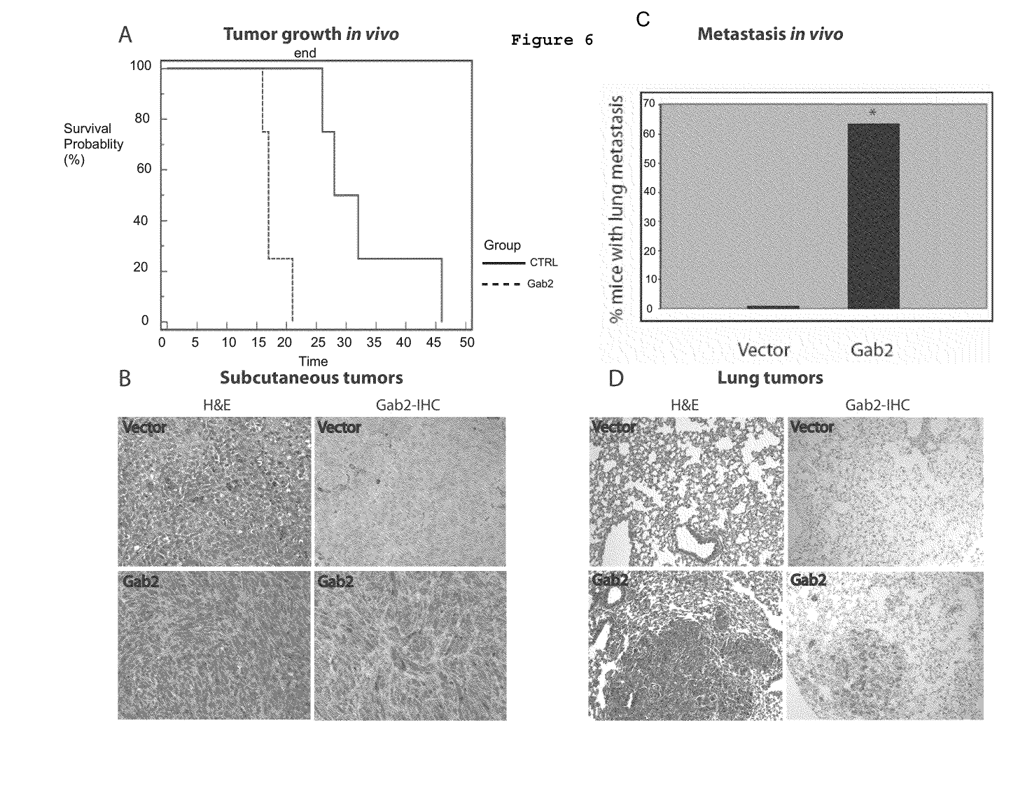

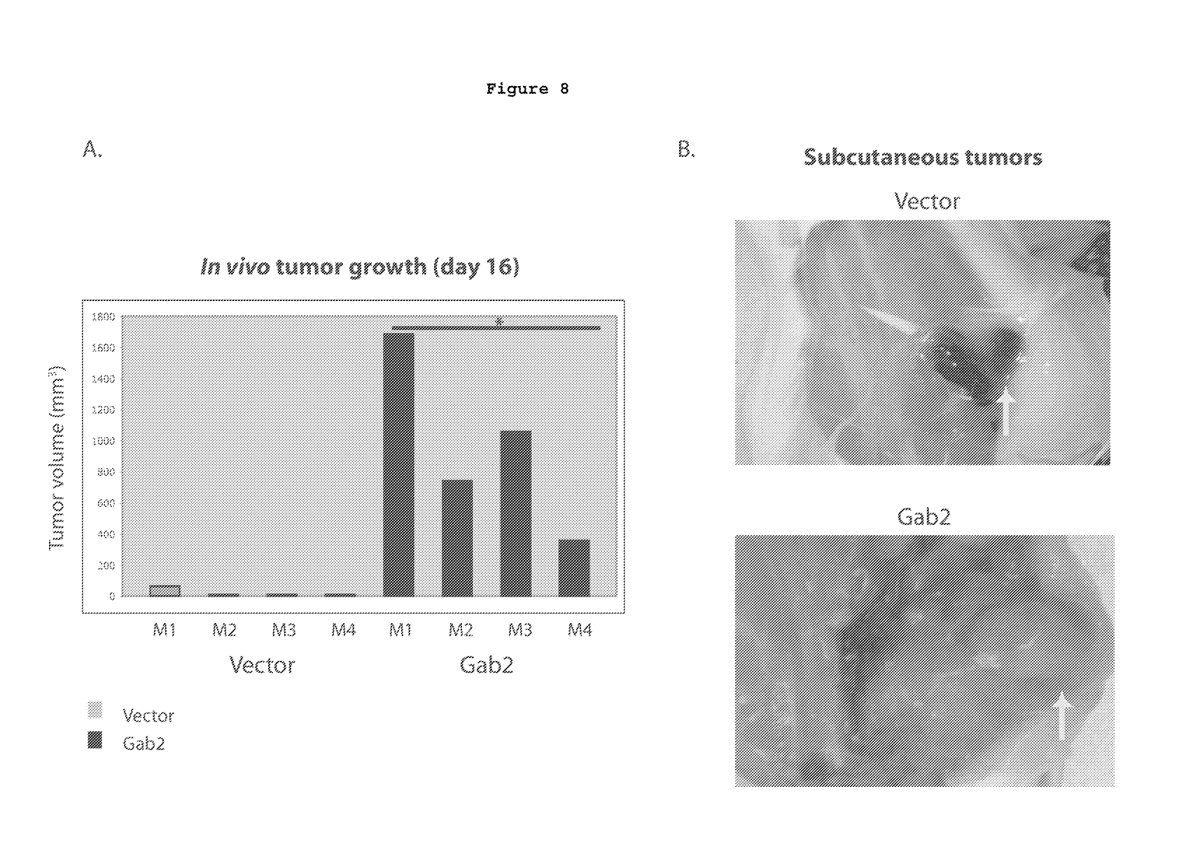

Gab2 Accelerates Tumorigenic Potential and Induces Metastasis in vivo. To study the effects of Gab2 on tumor growth in vivo, WM3862 primary melanoma cells with forced Gab2 expression or vector alone were injected subcutaneously into SCID mice. The mice were sacrificed when tumor surface area reached 1.5 cm². All mice injected with Gab2-overexpressing cells reached the tumor burden threshold between days 16 to 21, whereas mice injected with vector alone reached this threshold between days 26 to 46. The survival curves in FIG. 6A indicate that tumor growth was significantly increased in response to forced Gab2 expression suggesting a critical role for Gab2 in promoting tumor growth (FIG. 6A and 16, p<0.05). Intriguingly, WM3862 cells expressing vector alone led to pigmented tumors in SCID mice, whereas WM3862 cells overexpressing Gab2 were amelanotic (FIG. 8). It was then tested whether Gab2 activation confers a metastatic behavior in Gab2-overexpressing WM3862 cells in an experimental metastasis assay. Vector or Gab2-overexpressing cell lines were injected into SCID mice via the tail veins and animals were assessed for lung metastases three weeks after inoculation. One mouse in the Gab2-overexpressing group died prematurely and could not be evaluated. It was found tumor cells invading the lung parenchyma in seven out of 11 (63%) mice injected with Gab2-overexpressing WM3862 cells, whereas none of the mice injected with WM3862 cells expressing vector alone showed any evidence of lung metastases on multiple sections (FIG. 6B, p<0.05). These results suggest that Gab2 increases the intrinsic ability of melanoma cells to metastasize in vivo.

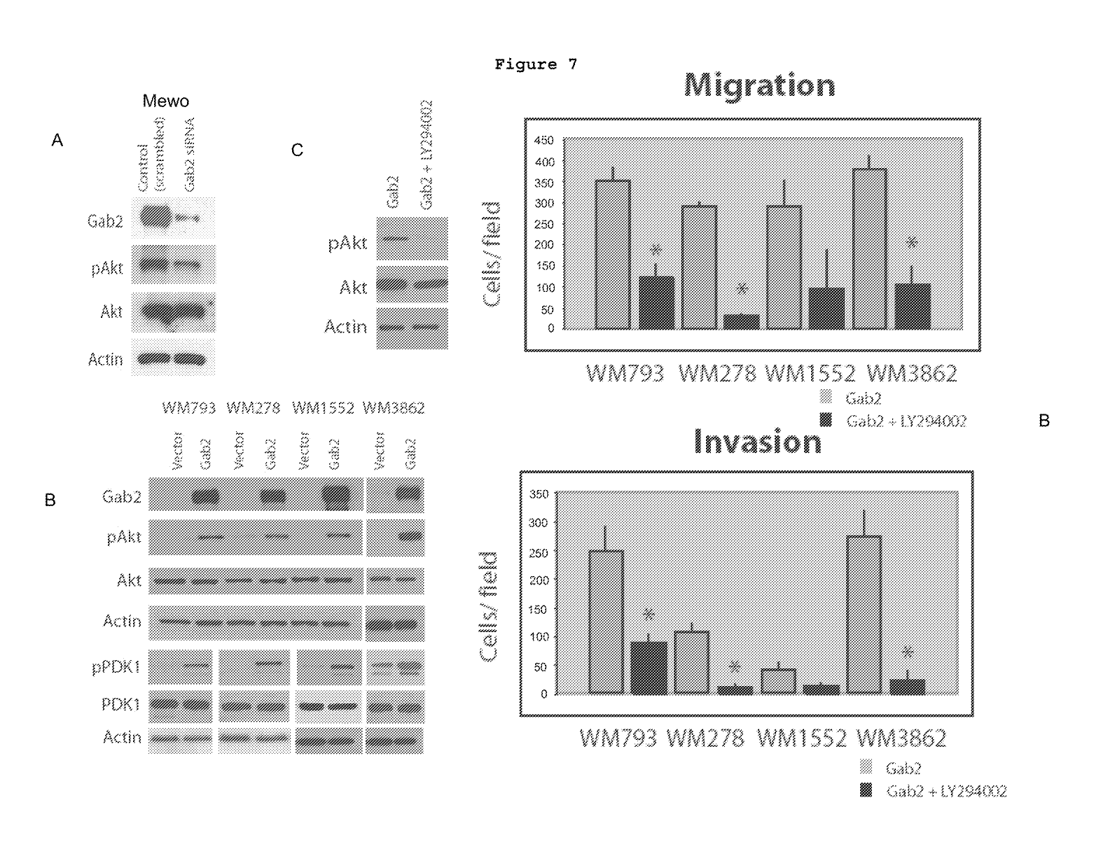

PI3K-Akt Signaling is Crucial for Gab2-Mediated Metastatic Phenotype. To gain mechanistic insights into signaling pathways that may contribute to Gab2mediated invasive phenotype in melanoma cells, AKT activation was examined. Silencing of Gab2 in a Gab2-overexpressing metastatic melanoma cell line, Mewo, resulted in a marked decrease in Akt phosphorylation (FIG. 7A). Furthermore, in a reciprocal experiment, forced expression of Gab2 into four primary melanoma cell lines with low endogenous Gab2 levels (WM793, WM278, WM1552, WM 3862, FIG. 3) increased Akt activation in the absence of exogenous growth factors (FIG. 7B), indicating that Gab2 activation leads to stimulation of Akt in melanoma cells. Since PDK1 is a downstream effector of PI3K that is recruited to the plasma membrane by phosphatidylinositol 3-phosphate (PIP3) and phosphorylates the activation loop of Akt and is critical for regulating cell migration, its activation in cells with ectopic Gab2 expression was examined. All melanoma cells with Gab2 overexpression also showed increased PDK1 phosphorylation (FIG. 7B), suggesting that Gab2 activation in melanoma cells may be resulting in Akt phosphorylation by signaling through PDK1.

To explore the possibility that Gab2 mediates the invasive phenotype via PI3KAkt signaling, the effect of the PI3K inhibitor, LY294002, on in vitro cell migration and invasion was examined. Inhibition of PI3K-Akt signaling in Gab2-overexpressing melanoma cells decreased migration and invasive capability significantly (p<0.05, FIG. 7C). Collectively, these data suggest that Gab2-mediated signaling results in Akt stimulation and promotes an invasive and metastatic phenotype in melanoma cells via PI3K/PDK1/Akt signaling.

### Discussion

Metastasis is a multistep process that requires the cell's ability to migrate and invade. In melanoma, both Erk and Akt activation are implicated in tumor cell migration and invasion. BRAFV600E and MEK activity are required for melanoma cell invasion in vitro. (16) Silencing of MMP-1, which is activated by Erk signaling, decreases metastatic capability of melanoma cells by inhibiting tumor cell collagenase activity and tumor-induced angiogenesis. (17) Mutant BRAF in melanoma cells disrupts actin cytoskeletal organization and focal adhesion dynamics through suppression of the Rho/ROCK/LIM kinase/cofilin pathway. (18) Blocking PI3K signaling in melanoma cells suppresses cell migration with a concomitant increase in E-cadherin. (19) Combined targeting of both RasErk and PI3K-Akt signaling pathways decreases invasiveness of melanoma cells suggesting a promising treatment strategy. (20) It is shown herein that Gab2 confers an invasive phenotype and drives progression of primary melanoma cells and enhances their metastatic capability via activation of the PI3K-Akt pathway. Gab2 expression correlates with clinical tumor progression in which higher levels of Gab2 are seen in metastatic melanomas. Collectively, data presented herein highlight the role of a previously unexplored molecule in melanoma, Gab2, in tumor progression and metastasis.

Alterations of both Ras-Erk and PI3K-Akt pathways are frequent in melanoma and occur in concert. (21) Gain-of-function mutations in NRAS or BRAF lead to Erk activation. (22) NRAS is mutated in 10-15% and BRAF is mutated in 50-70% of melanomas, and are mutually exclusive. (23) Co-expression of NRAS^{Q61R} and BRAF^{V600E} results in a senescent phenotype in melanoma cells, thus explaining the epistatic relationship due to selection against double mutant cells. (24) PTEN function is lost in between 5-20% of late stage melanomas leading to PI3K-Akt pathway activation (25) and Akt is overexpressed in up to 60% of melanomas. (26) NRAS mutations and PTEN inactivation occurs reciprocally in melanoma, which is thought to be due to activated NRAS being able to stimulate both Erk and Akt signaling pathways, and when present, not requiring PTEN deregulation. Ras transforms melanocytes more efficiently than BRAF possibly due to the PI3K component. (27) Gab2 can stimulate both Erk and Akt signaling through its interactions with Shp2 and p85 subunit of PI3K, respectively. In this study, the finding that Gab2 overexpression co-existed with BRAFV600E in several melanoma cell lines (Ht-144, Mel-1, A2058) and that Gab2 overexpression in primary melanoma cell lines with BRAFV600E resulted in increased invasiveness by activating Akt signaling, suggests that Gab2 may be cooperating with oncogenic BRAF for the acquisition of an invasive and metastatic phenotype in these cells. The relationship between increased DNA copy numbers of Gab2 and Gab2 overexpression in the context of NRAS and BRAF mutations and PTEN inactivation remains to be determined. 11q13-14 amplification is observed in several types of malignancy including melanoma, (28) breast (29,30) and ovarian cancer. (31) The amplification of this region was originally thought to involve a single amplicon spanning many megabases; however fine mapping of the region has identified four independent core regions. (29) Core 3 harbors cyclin D1, the overexpression of which is known to contribute to carcinogenesis of various tissue types including melanoma. (29,32) The amplification frequency of the cyclin D1 locus in primary melanoma is approximately 11% and is more frequent in acral melanoma subtype. (32) Down-regulation of cyclin D1 expression in melanoma cell lines reduces cell proliferation and induces apoptosis in vitro, and thus cyclin D1 has been suggested as an oncogene in melanoma driving the 11q13 amplicon. (32) Recently, Gab2 has been identified as a candidate for core 1 that contributes to breast cancer. (6) Studies provide evidence that amplification of Gab2 is a candidate mechanism for Gab2 overexpression in breast tumors with 11q13-14.1 amplification. (6) It is shown herein that increased copy numbers of Gab2 is seen in 11% of melanoma tumor samples and is independent of cyclin D1 locus, suggesting that the Gab2 region is not amplified as a passenger to cyclin D1 in melanoma. These results show that Gab2 is amplified in a subset of Gab2-overexpressing melanomas.

### REFERENCES

1. Jemal A, Siegel R, Ward E, Murray T, Xu J, Smigal C, Thun M J. Cancer statistics, 2006. CA Cancer J Clin 2006; 56:106-130.

2. Tsao H, Atkins M B, Sober A J. Management of cutaneous melanoma. N Engl J Med 2004; 351:998-1012.

3. Liu Y, Rohrschneider L R. The gift of Gab. FEBS Lett 2002; 515:1-7.

4. Gu H, Neel B G. The “Gab” in signal transduction. Trends Cell Biol 2003; 13:122 130.

5. Daly R J, Gu H, Parmar J, Malaney S, Lyons R J, Kairouz R, Head D R, Henshall S M, Neel B G, Sutherland R L. The docking protein Gab2 is overexpressed and estrogen regulated in human breast cancer. Oncogene 2002; 21:5175-5181.

6. Bentires-Alj M, Gil S G, Chan R, Wang Z C, Wang Y, Imanaka N, Harris L N, Richardson A, Neel B G, Gu H. A role for the scaffolding adapter GAB2 in breast cancer. Nat Med 2006; 12:114-121.

7. Bennett H L, Brummer T, Jeanes A, Yap A S, Daly R J. Gab2 and Src co-operate in human mammary epithelial cells to promote growth factor independence and disruption of acinar morphogenesis. Oncogene 2007

8. Ke Y, Wu D, Princen F, Nguyen T, Pang Y, Lesperance J, Muller W J, Oshima R G, Feng G S. Role of Gab2 in mammary tumorigenesis and metastasis. Oncogene 2007

9. Sattler M, Mohi M G, Pride Y B, Quinnan L R, Malouf N A, Podar K, Gesbert F, Iwasaki H, Li S, Van Etten R A, Gu H, Griffin J D, Neel B G. Critical role for Gab2 in transformation by BCR/ABL. Cancer Cell 2002; 1:479-492.

10. Valyi-Nagy I T, Hirka G, Jensen P J, Shih I M, Juhasz I, Herlyn M. Undifferentiated keratinocytes control growth, morphology, and antigen expression of normal melanocytes through cell-cell contact. Lab Invest 1993; 69:152-159.

11. Nowak N J, Miecznikowski J, Moore S, Gaile D, Bobadilla D, Smith D D, Kernstine K, Forman S J, Mhawech-Fauceglia P, Reid M, Stoler D, Loree T, Rigual N, Sullivan M, Weiss L M, Hicks D, Slovak M L. Challenges in array CGH for the analysis of cancer samples. Genetics in Medicine 2007

12. Zhao X, Weir B A, LaFramboise T, Lin M, Beroukhim R, Garraway L, Beheshti J, Lee J C, Naoki K, Richards W G, Sugarbaker D, Chen F, Rubin M A, Janne P A, Girard L, Minna J, Christiani D, Li C, Sellers W R, Meyerson M. Homozygous deletions and chromosome amplifications in human lung carcinomas revealed by single nucleotide polymorphism array analysis. Cancer Res 2005; 65:5561-5570.

13. Camp R L, Chung G G, Rimm D L. Automated subcellular localization and quantification of protein expression in tissue microarrays. Nat Med 2002; 8:13231327.

14. Levy L, Broad S, Zhu A J, Carroll J M, Khazaal I, Peault B, Watt F M. Optimised retroviral infection of human epidermal keratinocytes: long-term expression of transduced integrin gene following grafting on to SCID mice. Gene Ther 1998; 5:913-922.

15. Owens D M, Romero M R, Gardner C, Watt F M. Suprabasal alpha6beta4 integrin expression in epidermis results in enhanced tumourigenesis and disruption of TGFbeta signalling. J Cell Sci 2003; 116:3783-3791.

16. Huntington J T, Shields J M, Der C J, Wyatt C A, Benbow U, Slingluff C L, Jr., Brinckerhoff C E. Overexpression of collagenase 1 (MMP-1) is mediated by the ERK pathway in invasive melanoma cells: role of BRAF mutation and fibroblast growth factor signaling. J Biol Chem 2004; 279:33168-33176.

17. Blackburn J S, Rhodes C H, Coon C I, Brinckerhoff C E. RNA interference inhibition of matrix metalloproteinase-1 prevents melanoma metastasis by reducing tumor collagenase activity and angiogenesis. Cancer Res 2007; 67:10849-10858.

18. Klein R M, Spofford L S, Abel E V, Ortiz A, Aplin A E. B-RAF Regulation of Rnd3 Participates in Actin Cytoskeletal and Focal Adhesion Organization. Mol Biol Cell 2008; 19:498-508.

19. Stewart A L, Mhashilkar A M, Yang X H, Ekmekcioglu S, Saito Y, Sieger K, Schrock R, Onishi E, Swanson X, Mumm J B, Zumstein L, Watson G J, Snary D, Roth J A, Grimm E A, Ramesh R, Chada S. PI3 kinase blockade by Ad-PTEN inhibits invasion and induces apoptosis in RGP and metastatic melanoma cells. Mol Med 2002; 8:451-461.

20. Meier F, Busch S, Lasithiotakis K, Kulms D, Garbe C, Maczey E, Herlyn M, Schittek B. Combined targeting of MAPK and AKT signalling pathways is a promising strategy for melanoma treatment. Br J Dermatol 2007; 156:1204-1213.

21. Vivanco I, Sawyers C L. The phosphatidylinositol 3-Kinase AKT pathway in human cancer. Nat Rev Cancer 2002; 2:489-501.

22. Cohen C, Zavala-Pompa A, Sequeira J H, Shoji M, Sexton D G, Cotsonis G, Cerimele F, Govindarajan B, Macaron N, Arbiser J L. Mitogen-actived protein kinase activation is an early event in melanoma progression. Clin Cancer Res 2002; 8:3728-3733.

23. Gray-Schopfer V, Wellbrock C, Marais R. Melanoma biology and new targeted therapy. Nature 2007; 445:851-857.

24. Petti C, Molla A, Vegetti C, Ferrone S, Anichini A, Sensi M. Coexpression of NRASQ61R and BRAFV600E in human melanoma cells activates senescence and increases susceptibility to cell-mediated cytotoxicity. Cancer Res 2006; 66:6503-6511.

25. Wu H, Goel V, Haluska F G. PTEN signaling pathways in melanoma. Oncogene 2003; 22:3113-3122.

26. Stahl J M, Sharma A, Cheung M, Zimmerman M, Cheng J Q, Bosenberg M W, Kester M, Sandirasegarane L, Robertson G P. Deregulated Akt3 activity promotes development of malignant melanoma. Cancer Res 2004; 64:7002-7010.

27. Chudnovsky Y, Adams A E, Robbins P B, Lin Q, Khavari P A. Use of human tissue to assess the oncogenic activity of melanoma-associated mutations. Nat Genet 2005; 37:745-749.

28. Bastian B C, Kashani-Sabet M, Hamm H, Godfrey T, Moore D H, 2nd, Brocker E B, LeBoit P E, Pinkel D. Gene amplifications characterize acral melanoma and permit the detection of occult tumor cells in the surrounding skin. Cancer Res 2000; 60:1968-197

29. Ormandy C J, Musgrove E A, Hui R, Daly R J, Sutherland R L. Cyclin D1, EMS1 and 11q13 amplification in breast cancer. Breast Cancer Res Treat 2003; 78:323335.

30. Bekri S, Adelaide J, Merscher S, Grosgeorge J, Caroli-Bosc F, Perucca-Lostanlen D, Kelley P M, Pebusque M J, Theillet C, Birnbaum D, Gaudray P. Detailed map of a region commonly amplified at 11q13-->q14 in human breast carcinoma. Cytogenet Cell Genet 1997; 79:125-131.

31. Lambros M B, Fiegler H, Jones A, Gorman P, Roylance R R, Carter N P, Tomlinson I P. Analysis of ovarian cancer cell lines using array-based comparative genomic hybridization. J Pathol 2005; 205:29-40.

32. Sauter E R, Yeo U C, von Stemm A, Zhu W, Litwin S, Tichansky D S, Pistritto G, Nesbit M, Pinkel D, Herlyn M, Bastian B C. Cyclin D1 is a candidate oncogene in cutaneous melanoma. Cancer Res 2002; 62:3200-3206.

### EXAMPLE 2

### GAB2 Amplifications Refine Molecular Classification of Melanoma

Several melanoma subtypes are recognized based on anatomic site, sun exposure characteristics and histopathologic features. In recent years, the identification of distinct genetic aberrations among melanoma subtypes has resulted in improved classification, with the ultimate goal of developing treatment strategies based on molecular characteristics. However, identification of additional genetic events is necessary to refine the current melanoma classification and to develop novel therapeutic agents.

MAPK pathway is a key regulator of melanoma cell proliferation with ERK activation seen in majority of melanomas. BRAF and NRAS mutations, leading to constitutive activation of this pathway, occur in ˜50% and ˜15% of melanomas, respectively, and have been associated with melanomas arising from non-chronic sun exposed sites, most of which are located on trunk and extremities. Mutations in these genes are mutually exclusive in melanoma. In contrast, BRAF and NRAS mutations are rare in melanomas arising from sun protected areas such as acral, mucosal and uveal melanomas, suggesting genetic events or mechanisms other than oncogenic mutations in BRAF and NRAS leading to ERK activation in these melanoma subtypes. Amplification of the KIT locus on 4q12 and activating mutations in the KIT gene has recently been identified in a subset of melanomas (7% amplification and 3% mutation frequency) of which the majority consisted of melanomas on acral and mucosal sites.

GAB2 is a scaffolding protein that mediates interactions with various signaling pathways such as RAS-ERK and PI3K-AKT signaling. As shown herein, GAB2 is a critical molecule for melanoma tumor progression and metastasis by activating AKT signaling. Importantly, increased copy numbers and gene amplification of GAB2 were found in a subset of melanomas. In this example, clinical correlates of increased DNA copy numbers of GAB2 and its relation to genetic aberrations in BRAF, NRAS and KIT were examined.

### Patients and Methods

Study Population. Eighty five frozen melanoma tumor samples consisting of 23 cases arising from sun-protected sites (acral and mucous membranes) and 62 arising from head, neck, trunk and extremities were studied. Sixty-four cases were a part of a previously published data set. Clinical data such as gender, age at diagnosis, primary tumor depth, and primary tumor site were available for the majority of the cases. All patients, except two, had died of metastatic disease. The tumors were obtained from the archives of Fachklinik Hornheide and Columbia University. The study was approved by the Institutional Review Board of Columbia University.

Experimental Methods. Array based comparative genomic hybridization (array CGH) was performed using arrays with ˜19,000 RPCI-11 BAC clones providing an average resolution of 150 kb. Hybridization was carried out on 1 μg of genomic DNA, labeled by random priming, and analyzed as described previously. Mutation analysis was performed for the entire coding region of GAB2 and for the hot spot regions of BRAF (exon 15), NRAS (exons 2 and 3), and KIT (exons 11, 13, 17, and 18) using PCR-amplification. Purified PCR products were sequenced using a Big Dye Terminator cycle sequencing kit and an ABI Prism 310 automated sequencer system (Applied Biosystems, Foster City, Calif.).

Fluorescent in situ hybridization (FISH) was performed as described previously.¹² Briefly, RP11-653J20 and RP11-444N24 BAC clones were obtained from Invitrogen. DNA was prepared from BAC clones using standard methods and labeled by nick-translation using spectrum red or spectrum green dUTP fluorochromes. Spectrum red or spectrum green-labeled centromeric probes were used to enumerate chromosome numbers (Vysis, Downers Grove, Ill.). Hybridization signals were scored on at least 200 interphase nuclei from tissue sections on DAPI counterstained slides.

Immunohistochemistry was performed on paraffin-embedded tissue arrays and tissue sections using standard protocols with an antibody against GAB2 (26B6, Cell Signaling, Danvers, Mass.). Staining intensity levels were scored from 0 to 3, in which staining intensity of 2 or greater were considered positive. A sample with known positive staining was used as an external positive control. Lack of protein expression in the epidermis served as a negative control.

Statistical Methods. Simple linear regression was used for evaluating the relationship between continuous outcomes such as age, tumor depth and copy number change. Fisher's exact test and Pearson's X² test were used to assess the statistical significance of two categorical variables, such as the association of genetic alterations with primary tumor site. P values less than 0.05 were considered as significant.

### Results

Patient characteristics and the frequencies of genetic alterations in BRAF, NRAS, KIT, and GAB2 are summarized in Table 1. Based on previous literature, the primary tumor site was categorized into two groups; sun-protected sites including palms, soles, and mucous membranes; and sun-exposed sites including head, neck, trunk and extremities. There were 73% tumors arising from sun-exposed and 27% from sun-protected sites. All of the primary tumors were of intermediate thickness (1-4 mm) or thick (>4.0 mm).

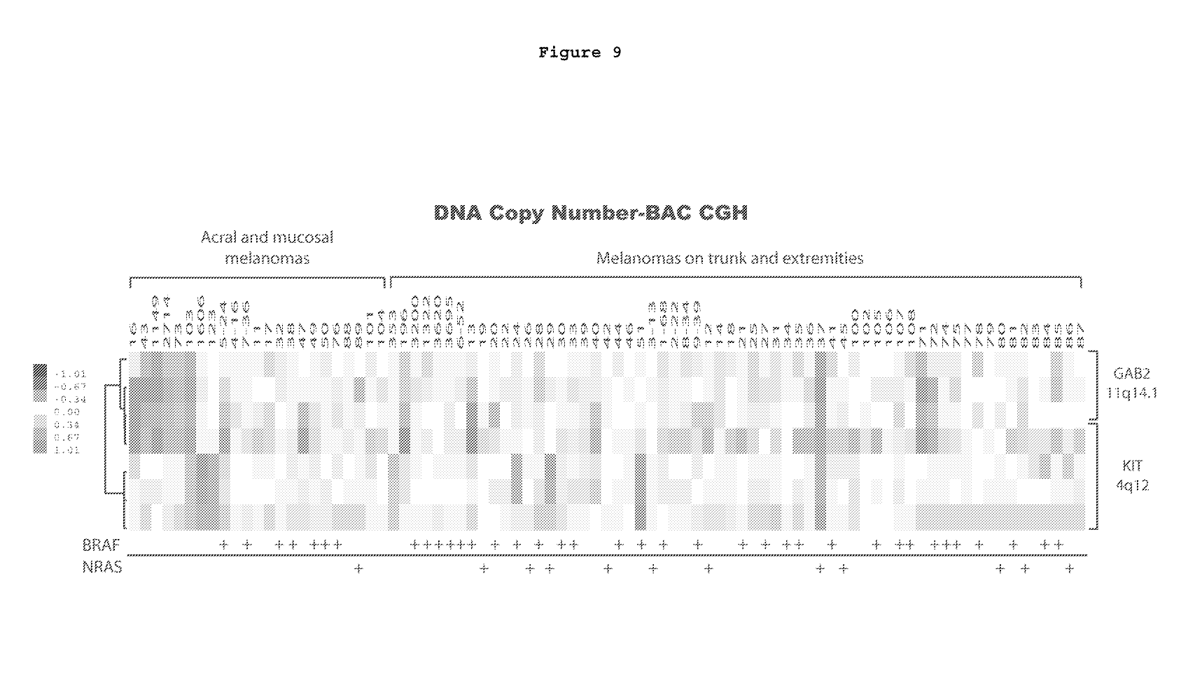

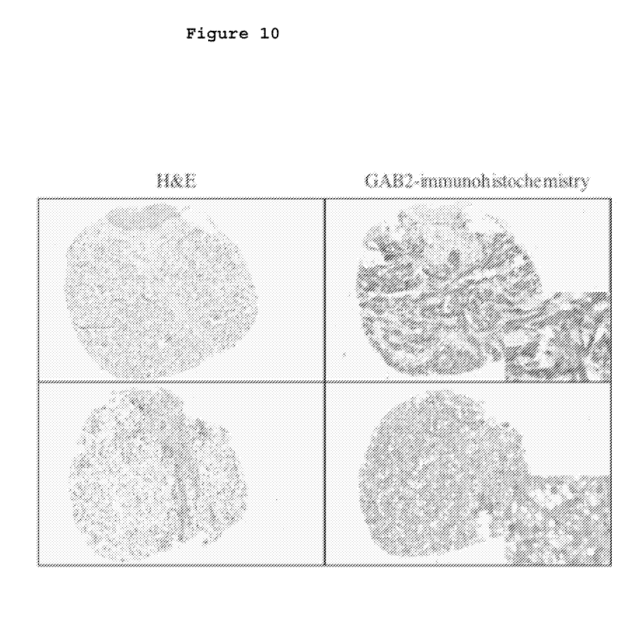

In an attempt to examine GAB2 amplifications in the context of other genetic events, a total of 85 melanoma tumor samples were evaluated for activating mutations in BRAF, NRAS, and KIT, and for copy number changes using array CGH. Mutations in BRAF and NRAS were mutually exclusive and the mutation frequencies were 43.5% (37/85) for BRAF and 14% (12/85) for NRAS. Increased copy numbers of the KIT locus on 4q12 were found in 5 of the 85 (6%) cases. GAB2 located on 11q14.1 was amplified in 8 of the 85 (9%) cases (Table 1, FIG. 9). Of the 8 cases with GAB2 amplifications, two cases co-existed with KIT amplifications, and two cases occurred together with a BRAF or an NRAS mutation. GAB2 amplifications occurred independent from KIT, BRAF, and NRAS in 5 of the 8 cases, all of which were melanomas arising from sun-protected sites (Table 2, FIG. 9). Mutation analysis of the hot spot regions of the KIT gene and the entire coding region of the GAB2 gene failed to detect any sequence variations. Increased copy numbers of the GAB2 locus were confirmed by FISH analysis. All amplified cases, except one case which could not be examined due to limited tissue, showed increased GAB2 protein expression by immunohistochemistry (Table 2, FIG. 10).

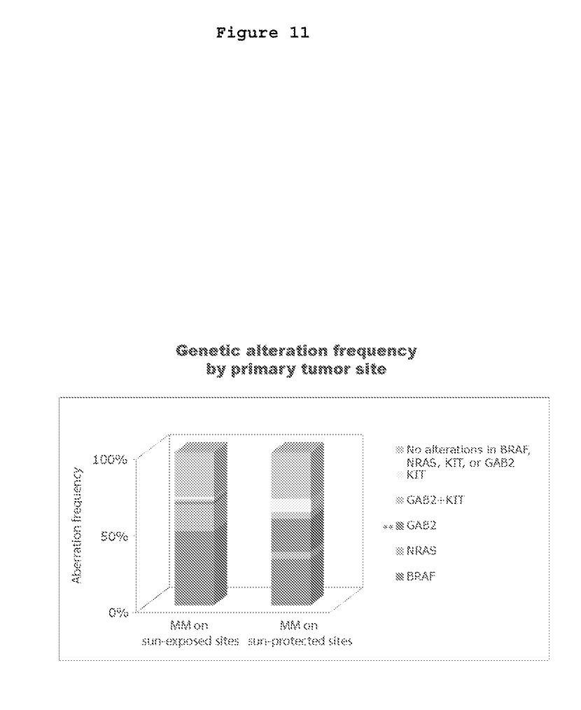

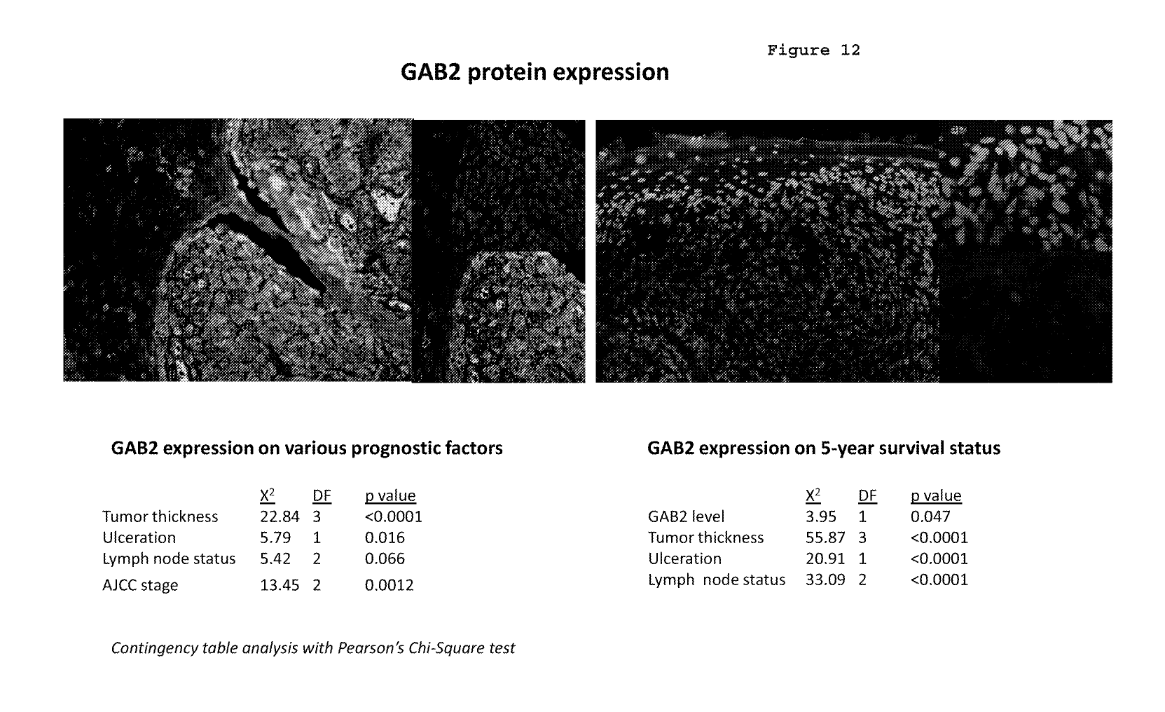

Genetic alterations in GAB2, BRAF, NRAS, and KIT in relation to clinical parameters such as primary tumor site were then examined. GAB2 amplifications were associated with melanomas arising from sun-protected sites (acral and mucous membranes) (P=0.005) (FIG. 11). Although there was limited number of cases with KIT amplification, they were more frequent, but not statistically significant, in melanomas arising from sun-protected sites compared to sun-exposed sites (P=0.135). Supervised hierarchical clustering of the melanomas by anatomic site showed clustering of GAB2 and KIT amplifications among the acral and mucosal melanomas (P=0.001, FIG. 9). BRAF and NRAS mutations were more frequently observed in melanomas occurring on sun-exposed sites when compared to sun-protected sites (P=0.016, FIG. 11). However, when analyzed independently, there was no significant correlation with BRAF or NRAS mutations and anatomic site. Similarly, an association with BRAF or NRAS mutations was not found when histopathologic classification (superficial spreading melanoma, nodular melanoma, and acral lentiginous melanoma) was applied to the dataset.

Among the 23 acral and mucosal melanomas studied, copy number changes in GAB2 were found in 26% (6/23) and KIT in 13% (3/23) of the cases (FIG. 11). Mutations in BRAF were identified in 30% (7/23) and NRAS in 4% (1/23) of the cases. None of these genetic alterations were found in the remaining 31%. Among the 62 melanomas arising from head and neck, trunk and extremities, mutations in BRAF or NRAS accounted for 72% of the cases (30/62 in BRAF, 11/62 in NRAS). GAB2 and KIT alterations were rare in this group each accounting for 3% (FIG. 11).

### Discussion

Melanomas arising from sun-protected sites represent a unique subset; BRAF and NRAS mutations are found at a lower frequency whereas genetic aberrations in KIT, CDK4 and CYCLIN D1 are observed at a higher frequency compared to melanomas occurring on other anatomic sites. In this study, it first highlights a novel subset of melanomas characterized by GAB2 amplifications and shows its association with melanomas arising from sun-protected sites. The finding that GAB2 amplifications occurred in acral and mucosal melanoma cases in the absence of mutations in BRAF and NRAS suggests that GAB2 may be critical in oncogenic transformation of a subset of BRAF/NRAS wild type melanomas. Second, this study implicates a role for GAB2 in refining molecular classification of melanomas and its potential use in therapeutic decision-making in the pharmacogenomics era. Finally, this study validates previous observations that genetic aberrations in acral and mucosal melanomas differ from other sites suggesting different mechanistic routes among melanoma subtypes.

Mutations and/or copy number increases in KIT are common among acral (36%) and mucosal (39%) melanomas. Imatinib, which inhibits tyrosine kinase activity of KIT, is currently being tested in clinical trials for melanomas that harbor gain-of-function mutations in KIT. Similarly, amplifications of CDK4 occur in a small subset of melanomas and are found frequently among acral and mucosal subtypes. Melanomas harboring CDK4 amplifications in the absence of mutations in BRAF or NRAS, but co-existing with KIT alterations have been described. In the present series of 85 melanomas only one case (1%, 1/85) with CDK4 locus amplification was found in which the primary tumor was located on the trunk. The present study is consistent with previous reports that CDK4 locus amplifications are observed in a small subset of melanomas. Amplification and overexpression of CYCLIN D1, located on 11q13.2, is well characterized in human tumors including melanoma. Increased copy numbers of CYCLIN D1 are found in 11% of primary melanomas and are found at higher frequency among acral melanomas (44.4%) compared to other melanoma subtypes. It has been previously reported that GAB2 amplification is observed independent of CYCLIN D1 locus in melanoma. Additional studies with larger cohorts of acral and mucosal melanomas can be used to validate genomic alterations in this subtype.

Although efforts in correlating genetic alterations with clinical correlates such as melanoma subtypes characterized by sun exposure characteristics provide insights into melanoma pathogenesis, it is likely that due to genetic heterogeneity such a correlation may not be perfect, and therefore the ultimate classification scheme will be based on molecular rather than clinical characteristics.

TABLE 1
Patient Characteristics And Frequencies Of Genetic Alterations

| Variable | Case (n = 85) |
|---|---|
| Median age at diagnosis, years | 54 |
| Gender, n | |
| Male | 40 |
| Female | 28 |
| DU | 17 |
| Primary tumor site, n | |
| Head and neck | 5 |
| Trunk and extremities | 57 |
| Acral and mucosal sites | 23 |
| Primary tumor thickness, n | |
| <1.0 mm | 0 |
| 1-4 mm | 35 |
| >4.0 mm | 35 |
| DU | 15 |
| Genetic alteration frequency, n (%) | |
| BRAF mutation | 37 (43.5) |
| NRAS mutation | 12 (14) |
| KIT amplification | 5 (6) |
| GAB2 amplification | 8 (9) |

Abbreviation: DU, data unavailable.

TABLE 2
GAB2 Amplified Cases By Array CGH

| | Anatomic | FISH | IHC | Mutations | | |
|---|---|---|---|---|---|---|
| Case no. | site | GAB2 | GAB2 | BRAF | NRAS | KIT |
| 16 | Acral | Amplified | Positive | WT | WT | WT |
| 43 | Acral | Amplified | Positive | WT | WT | WT |
| 1149 | Genitalia | Amplified | Positive | WT | WT | WT |
| 2714 | Acral | Amplified | Positive | WT | WT | WT |
| 73 | Acral | Amplified | Positive | WT | WT | WT |
| 103 | Acral | ND | ND | WT | WT | WT |
| 37 | Face | Amplified | Positive | WT | Q61K | WT |
| 72 | Leg | Amplified | Positive | WT | V600E | WT |

Abbreviations: FISH, fluorescent in situ hybridization; IHC, immunohistochemistry; WT, wild type; ND, not done due to limited tissue.

### EXAMPLE 3

### GAB2 Protein Expression

### Materials and Methods

In this study, the potential impact of Gab2 as a molecular prognostic marker for melanoma was examined using immunofluorescence analysis in a cohort of 128 patients with primary cutaneous melanoma. The study cohort is described in Table 3. Immunofluorescence staining was carried out on 5 μm paraffin-embedded tissue sections using anti-Gab2 (Cell Signaling, Danvers, Mass.; 26B6; 1:200 dilution) with standard protocols. Gab2 protein expression was graded as negative (none to weak staining) or positive (moderate to intense staining) by a pathologist. The clinical and histological attributes used in the prognostic factor analysis conducted by the American Joint Committee on Cancer (AJCC) were analyzed. For statistical analysis, Contingency table analysis with Pearson's Chi Square test is used. P value greater than 0.05 is considered significant.

### Results

High Gab2 expression was significantly correlated with increased tumor thickness (p<0.0001), ulceration (p=0.016), lymph node status (p=0.066), AJCC staging (p=0012), and 5-year survival status (p=0.047). These data validate the role of Gab2 in melanoma progression and metastasis, and highlight its potential as a novel prognostic marker.

The immunofluorescence studies examined Gab2 expression at the protein level. It was showed that Gab2 protein expression correlated with various known prognostic factors such as tumor thickness; which means increased chance of metastasis or poor prognosis. Gab2 protein expression also correlated with 5 year survival events, which means decreased chance of survival at 5 years.

TABLE 3
GAB2 Protein Expression

| Variable | N (%) | Median (range) |
|---|---|---|
| Age at diagnosis | | 59 (19-88) |
| Gender | | |
| Male | 67 (52) | |
| Female | 61 (48) | |
| Primary tumor site | | |
| Acral | 13 (10) | |
| Non-acral | 72 (56) | |
| Unknown | 43 (34) | |
| Tumor thickness | | |
| ≦1.0 mm | 39 (30) | |
| 1.0-4.0 mm | 51 (40) | |
| ≧4.0 mm | 38 (30) | |
| Tumor ulceration | | |
| Absent | 90 (70) | |
| Present | 38 (30) | |
| Events | | |
| Alive | 82 (64) | |
| Dead | 46 (36) | |
| Follow-up (mo.s) | | 74 (4-253) |
| Total | 128 | |

### REFERENCES

1. Curtin J A, Fridlyand J, Kageshita T, et al: Distinct sets of genetic alterations in melanoma. N Engl J Med 353:2135-47, 2005

2. Cohen C, Zavala-Pompa A, Sequeira J H, et al: Mitogen-actived protein kinase activation is an early event in melanoma progression. Clin Cancer Res 8:3728-33, 2002

3. Davies H, Bignell G R, Cox C, et al: Mutations of the BRAF gene in human cancer. Nature 417:949-54, 2002

4. Maldonado J L, Fridlyand J, Patel H, et al: Determinants of BRAF mutations in primary melanomas. J Natl Cancer Inst 95:1878-90, 2003

5. Takata M, Goto Y, Ichii N, et al: Constitutive activation of the mitogen-activated protein kinase signaling pathway in acral melanomas. J Invest Dermatol 125:318-22, 2005

6. Curtin J A, Busam K, Pinkel D, et al: Somatic activation of KIT in distinct subtypes of melanoma. J Clin Oncol 24:4340-6, 2006

7. Gu H, Neel B G: The “Gab” in signal transduction. Trends Cell Biol 13:122-30, 2003

8. Bentires-Alj M, Gil S G, Chan R, et al: A role for the scaffolding adapter GAB2 in breast cancer. Nat Med 12:114-21, 2006

9. Horst B, Gruvberger-Saal S, Hopkins B, et al: Gab2-mediated signaling promotes melanoma metastasis. Am J Pathol in press

10. Osoegawa K, Mammoser A G, Wu C, et al: A bacterial artificial chromosome library for sequencing the complete human genome. Genome Res 11:483-96, 2001

11. Nowak N J, Miecznikowski J, Moore S R, et al: Challenges in array comparative genomic hybridization for the analysis of cancer samples. Genet Med 9:585-95, 2007

12. Scotto L, Narayan G, Nandula S V, et al: Integrative genomics analysis of chromosome 5p gain in cervical cancer reveals target over-expressed genes, including Drosha. Mol Cancer 7:58, 2008

13. Curtin J A, Busam K, Pinkel D, et al: Somatic Activation of KIT in Distinct Subtypes of Melanoma. J Clin Oncol, 2006

14. Fecher L A, Cummings S D, Keefe M J, et al: Toward a molecular classification of melanoma. J Clin Oncol 25:1606-20, 2007

15. Muthusamy V, Hobbs C, Nogueira C, et al: Amplification of CDK4 and MDM2 in malignant melanoma. Genes Chromosomes Cancer 45:447-54, 2006

16. Smalley K S, Contractor R, Nguyen T K, et al: Identification of a novel subgroup of melanomas with KIT/cyclin-dependent kinase-4 overexpression. Cancer Res 68:5743-52, 2008

17. Sauter E R, Yeo U C, von Stemm A, et al: Cyclin D1 is a candidate oncogene in cutaneous melanoma. Cancer Res 62:3200-6, 2002

What is claimed is:

1. A method of detecting melanoma cells in a subject, comprising the steps of:
a) determining Gab2 (Grb2-associated binding protein 2) expression level in a tissue sample from the subject; and
b) comparing the Gab2 expression level to that in a tissue sample from a normal subject, wherein increased Gab2 expression would indicate presence of melanoma cells in the subject.

2. The method of claim 1, wherein the Gab2 expression level would further help determine the stage of melanoma and predict the subject's prognosis and outcome.

3. The method of claim 1, wherein increased Gab2 expression would indicate poor prognosis or poor chance of survival for the subject.

4. The method of claim 1, wherein the Gab2 expression level is higher in metastatic melanoma cells than primary melanoma cells.

5. The method of claim 1, wherein the tissue sample is skin biopsy sample.

6. The method of claim 1, wherein Gab2 expression is determined at the protein, mRNA or DNA level.

7. The method of claim 1, wherein increased Gab2 expression is selected from the group consisting of increased Gab2 protein expression, increased DNA copy number, and GAB2 gene amplification.

8. A method of inhibiting the development, progression, or metastasis of melanoma cells in a subject, comprising the step of administering to the subject an agent that decreases, inhibits or modifies Gab2 expression or cellular signaling mediated by Gab2, wherein decreased, inhibited or modified Gab2 expression or cellular signaling mediated by Gab2 would inhibit the development, progression, or metastasis of melanoma cells in the subject.

9. The method of claim 8, wherein Gab2 expression is inhibited or decreased at protein, mRNA or DNA level.

10. The method of claim 8, wherein the agent that inhibits or modifies Gab2 expression is an anti-Gab2 antibody, an anti-sense Gab2, a small interfering RNA, a small hairpin RNA, or microRNA against Gab2.

11. The method of claim 8, wherein signaling mediated by Gab2 is Ras-Erk signaling or PI3K-Akt signaling.

12. The method of claim 8, wherein the melanoma cells are invasive melanoma cells, metastatic melanoma cells, acral melanoma cells, or mucosal melanoma cells.

13. A method for identifying candidate anti-Gab2 agents which inhibit or modify the function of Gab2 protein, wherein said method comprises
a) obtaining a melanoma sample;
b) contacting candidate agents with the melanoma sample; and
c) assaying one or more activities mediated by Gab2 protein, wherein decreased Gab2-mediated activities in the presence of the candidate agents as compared to control indicates that the candidate agents are anti-Gab2 agents.

14. The method of claim 16, wherein the melanoma sample is a biopsy sample, invasive melanoma cells, or metastatic melanoma cells.

15. The method of claim 16, wherein the Gab2-mediated activity is Gab2-mediated tumor cell migration or invasive potential, Ras-Erk signaling, or PI3K-Akt signaling.

16. The method of claim 16, wherein the method is a high throughput screening method.
